(19) United States
(12) Patent Application Publication
(10) Pub. No.: US 2020/0331906 A1
(43) Pub. Date: Oct. 22, 2020

(54) COMPOUND, METHOD OF SYNTHESIZING COMPOUND WITH PYRIDINE RING AND LIGHT-EMITTING ELEMENT

(71) Applicant: Au Optronics Corporation, Hsinchu (TW)

(72) Inventors: Tzu-Hsien Yen, Hsinchu (TW); Yi-Hsin Lin, Hsinchu (TW); Chao-Wei Huang, Taichung City (TW)

(73) Assignee: Au Optronics Corporation, Hsinchu (TW)

(21) Appl. No.: 16/656,579

(22) Filed: Oct. 18, 2019

(30) Foreign Application Priority Data
Apr. 17, 2019 (TW) 108113339

(51) Int. Cl. C07D 471/04; H01L 51/00

(52) U.S. Cl. CPC C07D 471/04; H01L 51/5084; H01L 51/0052; H01L 51/0072

(57) ABSTRACT

A compound, a method of synthesizing a compound with pyridine ring, and a light-emitting element are provided. The compound has a structure represented by formula (1),

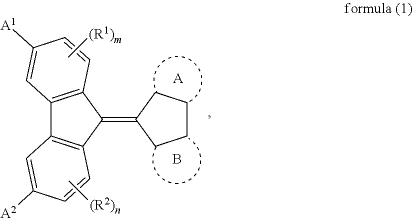

wherein ring A and ring B represent the same or different substituted or unsubstituted pyridine rings, respectively; A¹ and A² represent the same or different organic groups, respectively; R¹ and R² represent the same or different substituents, respectively; m and n represent 0, 1, 2 or 3, respectively.

### CROSS-REFERENCE TO RELATED APPLICATION

This application claims the priority benefit of Taiwan application serial no. 108113339, filed on Apr. 17, 2019. The entirety of the above-mentioned patent application is hereby incorporated by reference herein and made a part of this specification.

### BACKGROUND OF THE INVENTION

### Field of the Invention

The invention relates to a compound, a method of synthesizing a compound with a pyridine ring, and a light-emitting element.

### Description of Related Art

An organic light-emitting diode (OLED) or a quantum dot light-emitting diode (QLED) includes two metal electrodes and a light-emitting layer. In addition, the basic structure of the OLED or QLED further includes a hole transport layer and an electron transport layer. The anode generates a hole and the cathode generates an electron. The hole is transmitted via the hole transport layer and the electron is transmitted via the electron transport layer. The two are combined in a recombination region to form an exciton, and then emit light. However, the hole mobility in the hole transport layer and the electron mobility in the electron transport layer do not match, resulting in the recombination region being biased toward the hole transport layer or the electron transport layer. For example, the hole mobility is usually greater than the electron mobility, which in turn affects the performance and life of the light-emitting diode.

### SUMMARY OF THE INVENTION

The invention provides a compound which, when used as a material of an electron transport layer, allows the electron transport layer to have better electron mobility.

The invention provides a method of synthesizing a compound with a pyridine ring that may synthesize a compound suitable for an electron transport layer.

The invention provides a light-emitting element having good performance and life.

A compound of the invention has a structure represented by formula (1),

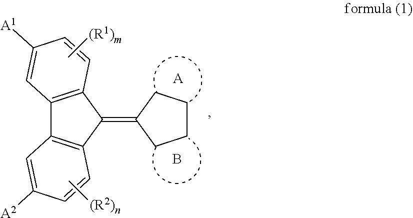

wherein ring A and ring B represent the same or different substituted or unsubstituted pyridine rings, respectively; A¹ and A² represent the same or different organic groups, respectively; R¹ and R² represent the same or different substituents, respectively; m and n represent 0, 1, 2 or 3, respectively.

A method of synthesizing a compound with a pyridine ring of the invention includes the following steps. A 9,10-phenanthrenequinone is converted to a borate ester compound. The borate ester compound and at least one compound containing a halogen functional group are reacted to form the compound with the pyridine ring. The borate ester compound has a structure represented by formula (1-1):

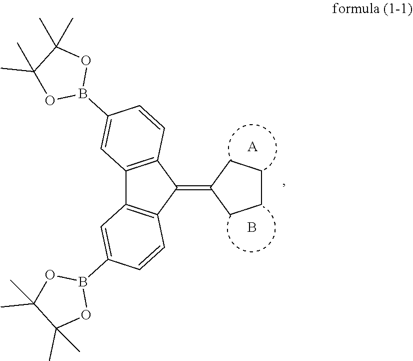

wherein ring A and ring B are the same or different substituted or unsubstituted pyridine rings, respectively.

A light-emitting element of the invention includes a first electrode, an organic layer, and a second electrode. The material of the organic layer includes the above compound or the compound prepared by the above method of synthesizing the compound with the pyridine ring. The organic layer is located between the first electrode and the second electrode.

Based on the above, the compound of an embodiment of the invention has a structure in which two fluorene rings are connected to each other by a double bond, wherein one carbon atom of each of the two benzene rings in one fluorene ring is substituted by a nitrogen atom. Therefore, the application of the above compound or the compound prepared by the above method of synthesizing the compound with the pyridine ring in the organic layer of the light-emitting element provides the organic layer with better electron mobility. In this way, the issue that the hole mobility in the hole transport layer and the electron mobility in the electron transport layer do not match may be alleviated, thereby improving the performance and life of the light-emitting element.

In order to make the aforementioned features and advantages of the disclosure more comprehensible, embodiments accompanied with FIGURES are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic view of a light-emitting element according to an embodiment of the invention.

### DESCRIPTION OF THE EMBODIMENTS

In the following, the invention is more comprehensively described with reference to FIGURES, and exemplary embodiments of the invention are shown in the FIGURES. As those skilled in the art will notice, the embodiments provided may be modified in various different manners without departing from the spirit or scope of the invention.

In the FIGURES, for clarity, the thicknesses of, for instance, layers, films, panels, and regions are enlarged. In the entire specification, the same reference numerals represent the same elements. It should be understood that, when a layer, film, region, or an element of a substrate is “on” another element or “connected to” another element, the element may be directly on the other element or connected to the other element, or an intermediate element may also be present. On the other hand, when an element is “directly on another element” or “directly connected to” another element, an intermediate element is not present. As used in the present specification, “connected to” may refer to a physical and/or electrical connection. Furthermore, “electrically connected” or “coupled” may mean that other elements are present between two elements.

In addition, relative terms such as “lower” or “bottom” and “upper” or “top” may be used herein to describe the relationship of one element to another element as shown in the FIGURE. It should be understood that relative terms are intended to encompass different orientations of the device in addition to the orientation shown. For example, if the device in one FIGURE is turned over, an element described as being on the “lower” side of other elements is oriented to being on the “upper” side of the other elements. Thus, the exemplary term “below” may include the orientations “below” and “above”, depending on the particular orientation of the FIGURE. Similarly, if the device in one FIGURE is turned over, an element described as “below” other elements or an element “below” is oriented “above” the other elements. Thus, the exemplary term “above” or “below” may encompass the orientations of above and below.

“About”, “similar”, or “substantially” used in the present specification include the value and the average value within an acceptable deviation range of a specific value confirmed by those having ordinary skill in the art, and the concerned measurement and a specific quantity (i.e., limitations of the measuring system) of measurement-related errors are taken into consideration. For instance, “about” may represent within one or a plurality of standard deviations of the value, or within 30%, 20%, ±10%, or 5%. Moreover, “about”, “similar”, or “substantially” used in the present specification may include a more acceptable deviation range or standard deviation according to optical properties, etching properties, or other properties, and one standard deviation does not need to apply to all of the properties.

Unless otherwise stated, all of the terminology used in the present specification (including technical and scientific terminology) have the same definition as those commonly understood by those skilled in the art of the invention. It should be further understood that, terminology defined in commonly-used dictionaries should be interpreted to have the same definitions in related art and in the entire specification of the invention, and are not interpreted as ideal or overly-formal definitions unless clearly stated as such in the present specification.

A compound according to the present embodiment has a structure represented by formula (1). Next, the structure represented by formula (1) is described in detail.

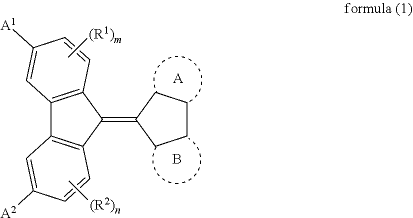

In formula (1), ring A and ring B are the same or different substituted or unsubstituted pyridine rings, respectively. When ring A and ring B are respectively pyridine rings, the induced electron-withdrawing effect thereof may reduce the lowest unoccupied molecular orbital (LUMO) of the compound to reduce the energy barrier of electron injection. Therefore, when applied to a light-emitting element, the performance of the light-emitting element may be improved. Ring A and Ring B may have a substituent, respectively. In the present embodiment, ring A and ring B preferably both have no substituents (that is, ring A and ring B are unsubstituted pyridine rings, respectively), so that the structure of the compound is flatter to achieve better molecular stacking; further, when the compound is used in an organic layer, better electron mobility may be achieved. R¹ and R² are respectively selected from the group consisting of an alkyl group, a cycloalkyl group, an aryl group, and a heteroaryl group. m and n are 0, 1, 2, or 3, respectively. In the present embodiment, m and n are preferably 0. When m and n are 0, the structure of the compound is flatter to achieve better molecular stacking; further, when the compound is used in an organic layer, better electron mobility may be achieved.

In formula (1), A¹ and A² are the same or different organic groups, respectively. In the present embodiment, A¹ and A² respectively include at least one of a substituted or unsubstituted benzene ring, a substituted or unsubstituted condensed aromatic hydrocarbon ring, a substituted or unsubstituted monocyclic aromatic heterocyclic ring, and a substituted or unsubstituted condensed aromatic heterocyclic ring. For example, at least one of A¹ and A² has a structure represented by formula (3).

X—Y—* formula (3)

In formula (3), X is a substituted or unsubstituted pyridine group or a substituted or unsubstituted pyrimidine group, Y is a substituted or unsubstituted divalent group, and * represents a bonding position. In the present embodiment, X is preferably

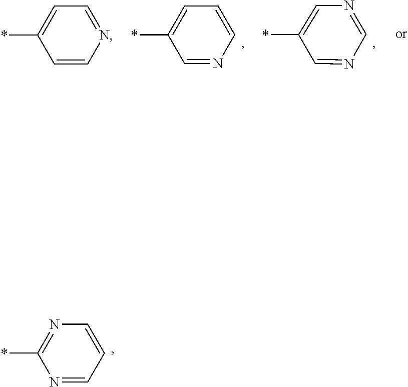

and * represents a bonding position. When X is an electron-withdrawing pyridine group or pyrimidine group, the structure of the compound may be flatter to achieve better molecular stacking. Moreover, X is preferably a pyrimidine group. Furthermore, compared to the compound in which X is a pyridine group, since the induced electron-withdrawing effect of the pyrimidine group is greater than the induced electron-drawing effect of the pyridine group, the highest occupied molecular orbital (HOMO) energy level and the lowest unoccupied molecular orbital (LUMO) energy level of the compound when X is a pyrimidine group are reduced more, and the energy barrier of electron injection is reduced. Therefore, when applied to a light-emitting element, the performance of the light-emitting element may be improved. In the present embodiment, Y is a substituted or unsubstituted divalent aromatic group, preferably a substituted or unsubstituted phenylene group, a substituted or unsubstituted pyridylene group, or a substituted or unsubstituted pyrimidinylene group. For example, Y may be a tolylene group to increase the triplet state energy of the compound. Compared with the structures of A¹ and A² respectively being X-* (i.e., without Y), when at least one of A¹ and A² has the structure represented by formula (3), the molecular weight of the compound may be increased to enhance the thermal stability thereof to increase lifetime. In the present embodiment, the substituent on X and the substituent on Y are respectively selected from the group consisting of an alkyl group, a cycloalkyl group, an aryl group, and a heteroaryl group.

In the present embodiment, formula (1) includes the structure represented by formula (2).

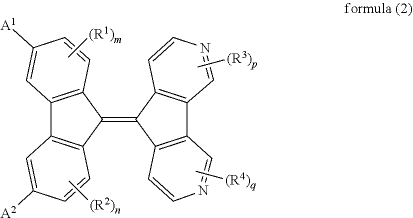

In formula (2), the definitions of A¹, A², R¹, and R² are the same as those of A¹, A², R¹, and R² in formula (1). The definitions of R³ and R⁴ are the same as the definitions of R¹ and R². R¹, R², R³, and R⁴ are respectively the same or different substituents. m, n, p, and q are 0, 1, 2, or 3, respectively. In the present embodiment, m, n, p, and q are preferably 0. When m, n, p, and q are 0, the structure of the compound is flatter to achieve better molecular stacking; further, when the compound is used in an organic layer, better electron mobility may be achieved. In the present embodiment, by substituting the carbon on the fluorene ring with a nitrogen atom, the highest occupied molecular orbital (HOMO) energy level and the lowest unoccupied molecular orbital (LUMO) energy level of the compound may be reduced, wherein when the nitrogen atom is substituted for carbon at the 3rd and 6th positions of the fluorene ring, a more significant induced electron-withdrawing effect may be achieved, such that the HOMO energy level and the LUMO energy level are more significantly reduced.

In the present embodiment, the compound represented by formula (1) is selected from one of a compound Et1 represented by formula (4-1), a compound Et2 represented by formula (4-2), a compound Et3 represented by formula (4-3), and a compound Et4 represented by formula (4-4).

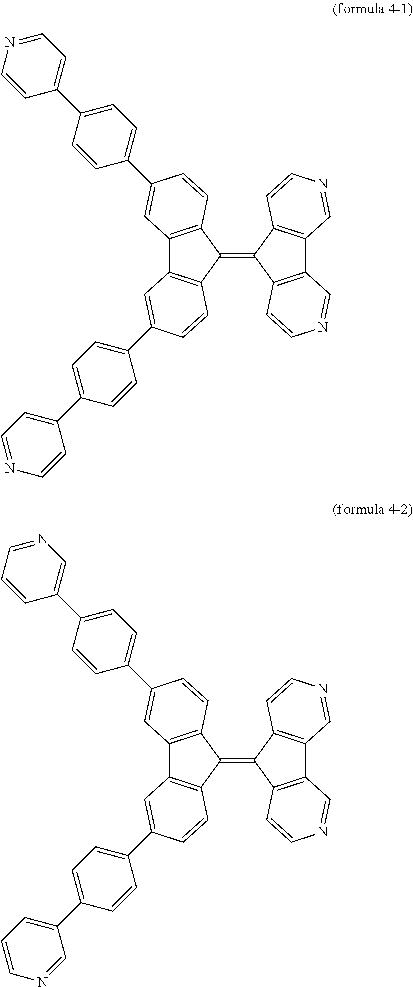 [SMILES: c1cc(-c2ccc(-c3ccc4c(c3)-c3cc(-c5ccc(-c6ccncc6)cc5)ccc3C4=C3c4ccncc4-c4cnccc43)cc2)ccn1 ; c1cncc(-c2ccc(-c3ccc4c(c3)-c3cc(-c5ccc(-c6cccnc6)cc5)ccc3C4=C3c4ccncc4-c4cnccc43)cc2)c1]

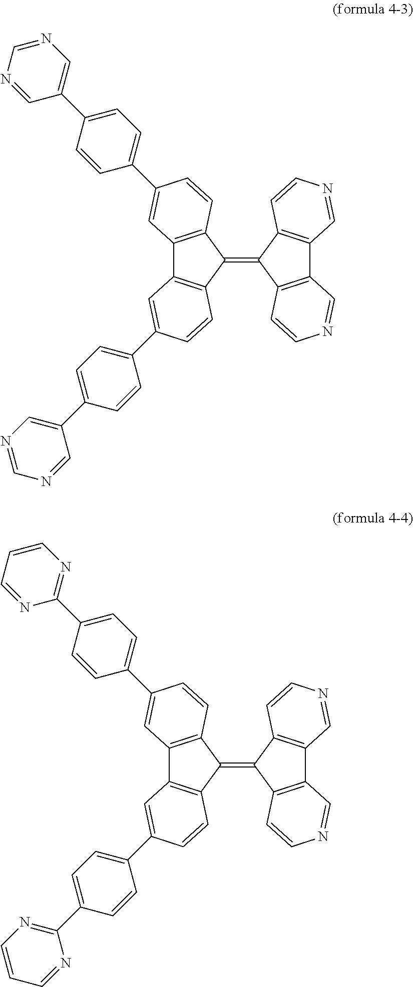 [SMILES: c1cc2c(cn1)-c1cnccc1C2=C1c2ccc(-c3ccc(-c4cncnc4)cc3)cc2-c2cc(-c3ccc(-c4cncnc4)cc3)ccc21 ; c1cnc(-c2ccc(-c3ccc4c(c3)-c3cc(-c5ccc(-c6ncccn6)cc5)ccc3C4=C3c4ccncc4-c4cnccc43)cc2)nc1]

Among all the above groups, hydrogen may be deuterium. Further, the alkyl group refers to, for example, a saturated aliphatic hydrocarbon group such as a methyl group, an ethyl group, an n-propyl group, an isopropyl group, an n-butyl group, a sec-butyl group, or a t-butyl group that may or may not have a substituent. The additional substituent at the time of substitution is not particularly limited, and examples may include: an alkyl group, an aryl group, and a heteroaryl group. This aspect is also common to the following description. In addition, the number of carbon atoms of the alkyl group is not particularly limited, but is preferably in the range of 1 or more and 20 or less, and more preferably in the range of 1 or more and 8 or less, from the viewpoint of availability or cost.

The cycloalkyl group refers to, for example, a saturated alicyclic hydrocarbon group such as a cyclopropyl group, a cyclohexyl group, a norbornyl group, or an adamantyl group that may or may not have a substituent. The number of carbon atoms of the cycloalkyl group is not particularly limited, but is preferably in the range of 3 or more and 20 or less.

The aryl group refers to, for example, an aromatic hydrocarbon group such as a phenyl group, a naphthyl group, a biphenyl group, a phenanthrene group, a diphenylbenzene group, a pyrene group, or a fluoranthene group. The aryl group may or may not have a substituent. The number of carbon atoms of the aryl group is not particularly limited, but is preferably in the range of 6 or more and 40 or less.

The heteroaryl group refers to a cyclic aromatic group having an atom other than carbon in one or more rings, such as a furyl group, a thiophenyl group, a pyridine group, a quinoline group, an isoquinoline group, a pyrazine group, a pyrimidine group, a naphthyridines group, a benzofuran group, a benzothiophenyl group, an indole group, a dibenzofuran group, a dibenzothiophenyl group, or a carbazole group that may be unsubstituted or substituted. The number of carbon atoms of the heteroaryl group is not particularly limited, but is preferably in the range of 2 or more and 30 or less.

The halogen refers to an atom selected from fluorine, chlorine, bromine, and iodine.

Examples of the condensed aromatic hydrocarbon ring may include, for example: a naphthalene ring, an azulene ring, an anthracycline, a phenanthrene ring, a pyrene ring, a chrysene ring, a naphthacene ring, a triphenylene ring, an acenaphthene ring, a coronene ring, a fluorene ring, a fluoranthene ring, a pentacene ring, a perilene ring, a pentaphene ring, a picene ring, a pyranthrene ring, an anthanthrene ring, and the like. Moreover, the condensed aromatic hydrocarbon ring may have a substituent.

Examples of the monocyclic aromatic heterocyclic ring may include a furan ring, a thiophene ring, a pyridine ring, a pyridazine ring, a pyrimidine ring, a pyrazine ring, an oxadiazole ring, a triazole ring, an imidazole ring, a pyrazole ring, a thiazole ring, and the like. Moreover, the monocyclic aromatic heterocyclic ring may have a substituent.

Examples of the condensed aromatic heterocyclic ring may include a quinoline ring, an isoquinoline ring, a quinoxaline ring, a benzimidazole ring, an indole ring, a benzothiazole ring, a benzoxazole ring, a quinazoline ring, a phthalazine ring, a carbazole ring, a carboline ring, a diazacarbazole ring (indicating one of the carbon atoms constituting a hydrocarbon ring of the carboline ring is further substituted by a nitrogen atom), and the like. Moreover, the condensed aromatic heterocyclic ring may have a substituent.

The method of synthesizing the compound with the pyridine ring according to the present embodiment includes the steps of: converting 9,10-phenanthrenequinone to a borate ester compound; and reacting the borate ester compound and a compound containing a halogen functional group to form the compound with the pyridine ring. The borate ester compound has a structure represented by formula (1-1).

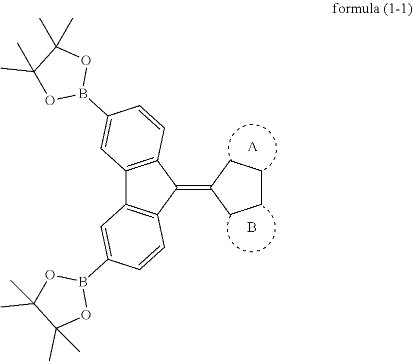

In formula (1-1), ring A and ring B are the same or different substituted or unsubstituted pyridine rings, respectively. In the present embodiment, the compound containing the halogen functional group includes at least one of

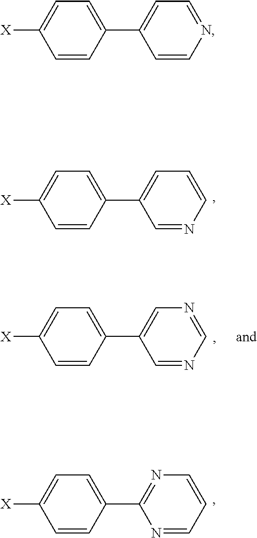

X is a halogen atom, such as an atom selected from fluorine, chlorine, bromine, and iodine. In the present embodiment, the borate ester compound and the compound containing the halogen functional group are reacted. However, the invention is not limited thereto. In other embodiments, the borate ester compound may also be first reacted with a compound containing a halogen functional group, and then reacted with another compound containing a halogen functional group to form a compound in which A¹ and A² are different organic groups in chemical formula (1).

In some embodiments, the method of synthesizing the compound having the pyridine ring further includes the following steps: converting 9,10-phenanthrenequinone to a substituted or unsubstituted fluorenone; and converting the substituted or unsubstituted fluorenone to the borate ester compound of formula (1-1).

Exemplary embodiments of the invention are now described in detail with reference to examples. However, the following examples are for illustrative purposes only, and the scope of the present specification is not limited to the scope of the examples and includes the scopes described in the appended claims and their substitutions and modifications.

### Synthesis Example 1: Prepare 3,6-dibromo-9H-fluoren-9-one

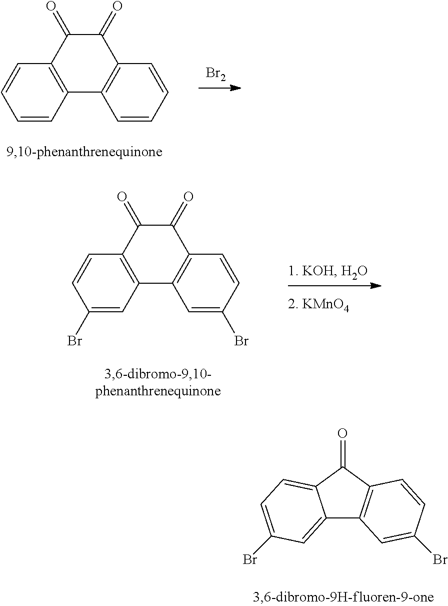

4.2 g (20 mmol) of 9,10-phenanthrenequinone and 2.25 mL (88 mmol) of bromine were reacted in a bromination reaction at a reaction temperature of 130° C. for 16 hours to form 3,6-dibromo-9,10-phenanthrenequinone. Next, after 7.32 g (130 mmol) of potassium hydroxide and water were mixed into a solution with a concentration of 1.6 M, the solution and 14.6 g (91.4 mmol) of potassium permanganate (KMnO₄) were added in a beaker having the 3,6-dibromo-9,10-phenanthrenequinone in order and reacted at 130° C. for 1 hour. The solvent was removed in vacuum, and 3.2 g (9.5 mmol) of 3,6-dibromo-9H-fluoren-9-one was obtained via column chromatography of the crude compound.

### Synthesis Example 2: Prepare 3,6-diazafluoren-9-thione

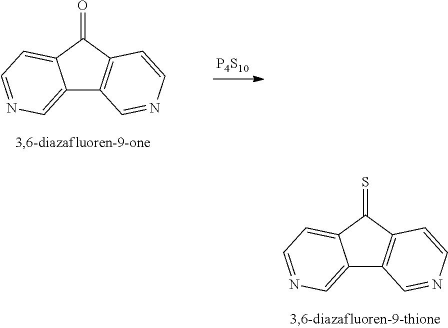

1.8 g (10 mmol) of 3,6-diazafluoren-9-one and 8.9 g (20 mmol) of phosphorus pentasulfide (P₄S₁₀) were added to 200 ml of toluene and reacted at 130° C. for 16 hours. The solvent was removed in vacuum, and 1.8 g (9 mmol) of 3,6-diazafluoren-9-thione was obtained via column chromatography of the crude compound.

### Synthesis Example 3: Prepare Borate Ester Compound of Formula (2-2)

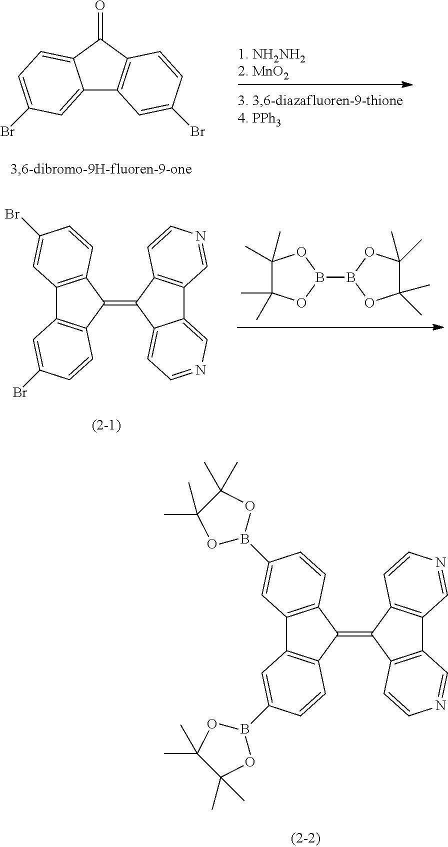

3.2 g (9.5 mmol) of 3,6-dibromo-9H-fluoren-9-one was added to 4.7 ml of hydrazine aqueous solution (NH₂NH_{2(aq)}) and reacted at 60° C. for 0.5 hours to form a hydrazone compound. Next, 0.8 g (9.5 mmol) of manganese dioxide (MnO₂) was added and reacted at room temperature for 48 hours such that the hydrazone compound was reacted in an oxidization reaction to form a diazo compound. Next, 1.8 g (9 mmol) of 3,6-diazafluoren-9-thione and 2.4 g (9 mmol) of triphenylphosphine (PPh₃) were added to perform a Barton-Kellogg reaction for 1 hour to form the compound represented by formula (2-1). Next, 5.0 g (19.8 mmol) of bis(pinacolato)diboron was added to perform a Suzuki Coupling reaction with the compound represented by formula (2-1) under the catalysis of a palladium metal (Pd) at 80° C. for 48 hours. The solvent was removed in vacuum, and 4.5 g (8 mmol) of the borate ester compound represented by formula (2-2) was obtained via column chromatography of the crude compound.

### Preparation Example 1: Prepare Compound Et1 Represented by Formula (4-1)

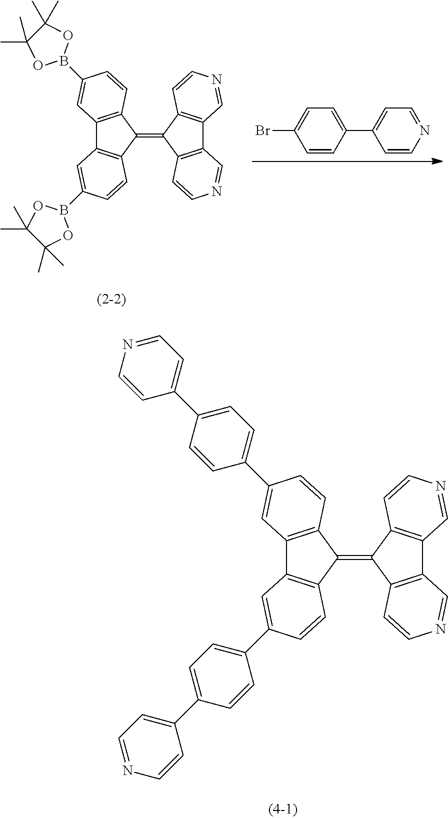 [SMILES: CC1(C)OB(c2ccc3c(c2)-c2cc(B4OC(C)(C)C(C)(C)O4)ccc2C3=C2c3ccncc3-c3cnccc32)OC1(C)C ; Brc1ccc(-c2ccncc2)cc1 ; c1cc(-c2ccc(-c3ccc4c(c3)-c3cc(-c5ccc(-c6ccncc6)cc5)ccc3C4=C3c4ccncc4-c4cnccc43)cc2)ccn1]

4.5 g (8 mmol) of the borate ester compound represented by formula (2-2) and 5.61 g (24 mmol) of 4-(4-bromophenyl)pyridine were added to 120 ml of toluene to perform a Suzuki Coupling reaction under the catalysis of a palladium metal (Pd) at 120° C. for 48 hours. The solvent was removed in vacuum, and 4.1 g (6.4 mmol) of the compound Et1 (yield: 80%) represented by formula (4-1) was obtained via column chromatography of the crude compound.

MS[M+H]⁺=636.2310

### Preparation Example 2: Prepare Compound Et2 Represented by Formula (4-2)

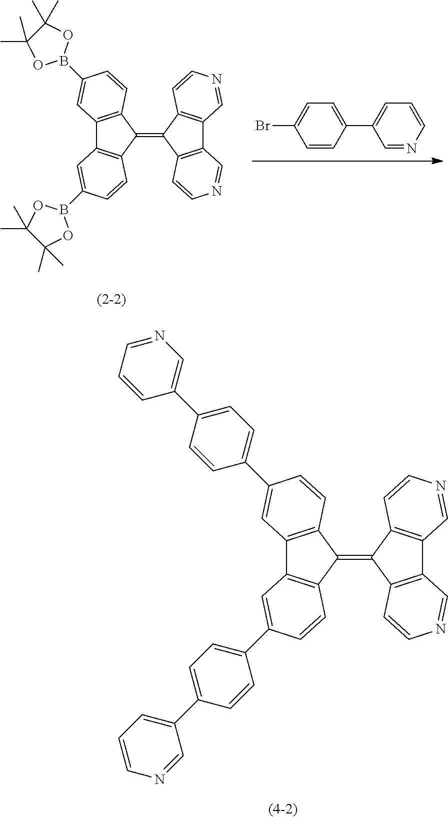 [SMILES: CC1(C)OB(c2ccc3c(c2)-c2cc(B4OC(C)(C)C(C)(C)O4)ccc2C3=C2c3ccncc3-c3cnccc32)OC1(C)C ; Brc1ccc(-c2cccnc2)cc1 ; c1cncc(-c2ccc(-c3ccc4c(c3)-c3cc(-c5ccc(-c6cccnc6)cc5)ccc3C4=C3c4ccncc4-c4cnccc43)cc2)c1]

4.5 g (8 mmol) of the borate ester compound represented by formula (2-2) and 5.61 g (24 mmol) of 3-(4-bromophenyl)pyridine were added to 120 ml of toluene to perform a Suzuki Coupling reaction under the catalysis of a palladium metal (Pd) at 120° C. for 48 hours. The solvent was removed in vacuum, and 4.1 g (6.4 mmol) of the compound Et2 (yield: 80%) represented by formula (4-2) was obtained via column chromatography of the crude compound.

MS[M+H]⁺=636.2351

### Preparation Example 3: Prepare Compound Et3 Represented by Formula (4-3)

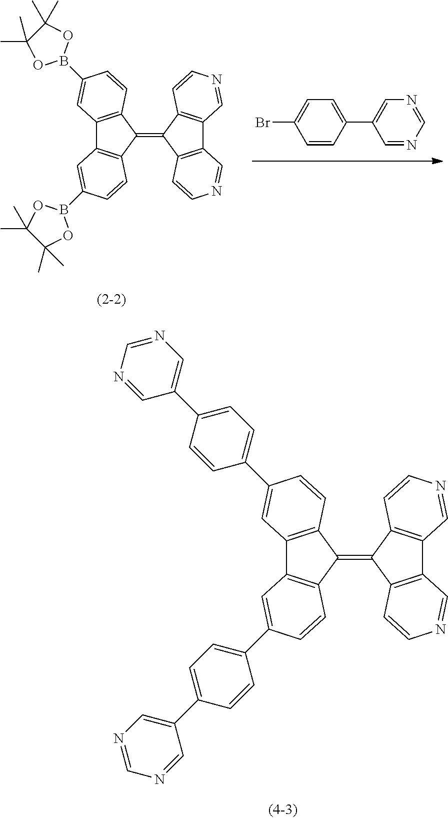 [SMILES: CC1(C)OB(c2ccc3c(c2)-c2cc(B4OC(C)(C)C(C)(C)O4)ccc2C3=C2c3ccncc3-c3cnccc32)OC1(C)C ; Brc1ccc(-c2cncnc2)cc1 ; c1cc2c(cn1)-c1cnccc1C2=C1c2ccc(-c3ccc(-c4cncnc4)cc3)cc2-c2cc(-c3ccc(-c4cncnc4)cc3)ccc21]

4.5 g (8 mmol) of the borate ester compound represented by formula (2-2) and 5.64 g (24 mmol) of 5-(4-bromophenyl)pyrimidine were added to 120 ml of toluene to perform a Suzuki Coupling reaction under the catalysis of a palladium metal (Pd) at 120° C. for 48 hours. The solvent was removed in vacuum, and 3.4 g (5.36 mmol) of the compound Et3 (yield: 67%) represented by formula (4-3) was obtained via column chromatography of the crude compound.

MS[M+H]⁺=638.2222

### Preparation Example 4: Prepare Compound Et4 Represented by Formula (4-4)

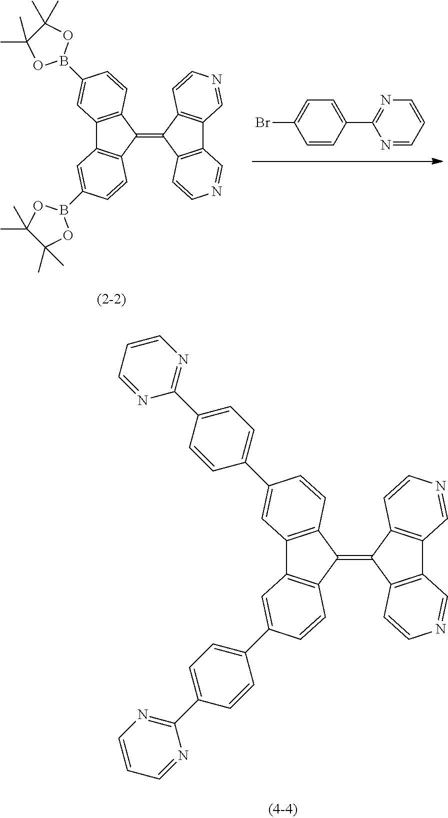 [SMILES: CC1(C)OB(c2ccc3c(c2)-c2cc(B4OC(C)(C)C(C)(C)O4)ccc2C3=C2c3ccncc3-c3cnccc32)OC1(C)C ; Brc1ccc(-c2ncccn2)cc1 ; c1cnc(-c2ccc(-c3ccc4c(c3)-c3cc(-c5ccc(-c6ncccn6)cc5)ccc3C4=C3c4ccncc4-c4cnccc43)cc2)nc1]

4.5 g (8 mmol) of the borate ester compound represented by formula (2-2) and 5.64 g (24 mmol) of 2-(4-bromophenyl)pyrimidine were added to 120 ml of toluene to perform a Suzuki Coupling reaction under the catalysis of a palladium metal (Pd) at 120° C. for 48 hours. The solvent was removed in vacuum, and 3.4 g (5.36 mmol) of the compound Et4 (yield: 67%) represented by formula (4-4) was obtained via column chromatography of the crude compound.

MS[M+H]⁺=638.2208

<Computation of Physical Properties of Compound Via Quantum Chemistry>

The highest occupied molecular orbital (HOMO) energy level, the lowest unoccupied molecular orbital (LUMO) energy level, and the energy level difference related to the synthesized compound Et1, compound Et2, compound Et3, and compound Et4 are obtained via quantum chemical calculations performed by the quantum chemical calculation program Gaussian 09 written by American Gaussian, Inc. In the calculation, Density Functional Theory (DFT) is used, and B3LYP is used as a functional and 6-31G* is used as a basis set to calculate the most stable structure of each compound under ground state under vacuum and the HOMO/LUMO energy level thereof. The calculation results are shown in Table 1.

TABLE 1

| | Et1 | Et2 | Et3 | Et4 |
|---|---|---|---|---|
| HOMO (eV) | −6.2 | −6.1 | −6.3 | −6.5 |
| LUMO (eV) | −3.0 | −2.8 | −3.1 | −3.2 |
| Energy gap | 3.2 | 3.3 | 3.2 | 3.3 |
| (eV) | | | | |

As shown in Table 1, the HOMO energy levels of the compound Et1, the compound Et2, the compound Et3, and the compound Et4 are between −6.5 eV and −6.1 eV, the LUMO energy levels thereof are between −3.2 eV and −2.8 eV, and the energy level differences thereof are between 3.2 eV and 3.3 eV. Compared with a material commonly used for an electron transport layer having a LUMO energy level of about −3.0 to −2.7 eV, a HOMO energy level of about −6.4 to −6.0 eV, and an energy level difference of about 3.4 to 3.8 eV, the compounds in the embodiments of the invention have lower LUMO energy level and HOMO energy level. When the compound has a lower LUMO energy level, the energy barrier of electron injection may be reduced. Therefore, the above compounds are suitable for use in a light-emitting element.

Next, a light-emitting element using the above compound as an organic layer is described.

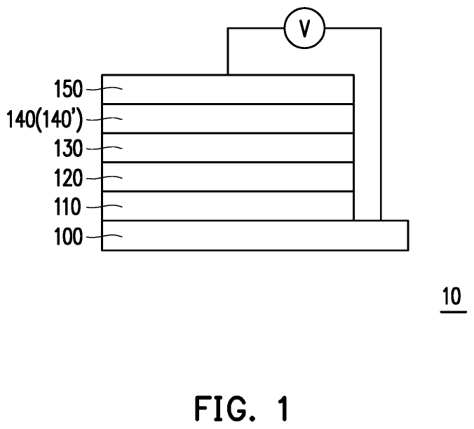

FIG. 1 is a schematic view of a light-emitting element according to an embodiment of the invention. A light-emitting element 10 includes a first electrode 110, an organic layer 140, and a second electrode 150. The second electrode 150 is disposed opposite to the first electrode 100. The material of the first electrode 100 and the second electrode 150 may be a transparent conductive material, a translucent conductive material, or an opaque conductive material. The first electrode 100 and the second electrode 150 may be a single layer or multilayer structure. The transparent conductive material may include a metal oxide such as indium tin oxide, indium zinc oxide, aluminum tin oxide, aluminum zinc oxide, indium germanium zinc oxide, other suitable oxides (such as zinc oxide), or stacked layers of at least two of the above. The opaque conductive material includes a metal such as gold, silver, aluminum, molybdenum, copper, titanium, chromium, tungsten, or other suitable metals. The translucent conductive material includes an extremely thin metal, such as a metal thin film having a thickness of less than 500 nanometers, or other translucent materials having a low work function. In the present embodiment, the first electrode 100 is, for example, an anode, and the second electrode 150 is, for example, a cathode. However, it should be mentioned that the first electrode 100 may also be the cathode and the second electrode 150 may be the anode depending on design requirements.

The organic layer 140 is located between the first electrode 100 and the second electrode 150. The material of the organic layer 140 includes the above compound or the compound prepared by the above method of synthesizing the compound with the pyridine ring. In the present embodiment, the organic layer 140 includes an electron transport layer 140′. When the material of the electron transport layer 140′ includes the above compound or the compound prepared by the above method of synthesizing the compound with the pyridine ring, the electron mobility of the electron transport layer 140′ is between 1×10−4 cm²V⁻¹s⁻¹ and 9×10−4 cm²V⁻¹ s⁻¹.

In the present embodiment, the light-emitting element 10 further includes a hole injection layer 110. The hole injection layer 110 is located between the organic layer 140 and the first electrode 100. The material of the hole injection layer 110 may be an organic material or an inorganic material. The organic material is, for example, poly(3,4-ethylenedioxythiophene) (PEDOT), poly(styrenesulfonate) (PSS), or a combination thereof (PEDOT:PSS). The carrier mobility of the hole injection layer 110 is, for example, about 4.5×10−2 cm²V⁻¹s⁻¹. However, the invention is not limited thereto. In other embodiments, the material of the hole injection layer 110 may be selected from other suitable materials.

In the present embodiment, the light-emitting element 10 further includes a hole transport layer 120. The hole transport layer 120 is located between the hole injection layer 110 and the organic layer 140. In the present embodiment, the hole mobility of the hole transport layer 120 is between 1×10⁻⁴ cm²V⁻¹s⁻¹ and 9×10⁻⁴ cm²V⁻¹s⁻¹. When the hole mobility of the hole transport layer 120 and the electron mobility of the electron transport layer 140′ are close, the issue that the hole mobility in the hole transport layer 120 and the electron mobility in the electron transport layer 140′ do not match may be alleviated. For example, the material of the hole transport layer 120 may be an organic material or an inorganic material. The organic material is, for example, poly(9,9′-dioctylfluorene-co-N-(4-butylphenyl) diphenylamine) (TFB), poly(N-vinylcarbazole) (PVK), or other suitable materials. However, the invention is not limited thereto. In other embodiments, the material of the hole transport layer 120 may be selected such that the hole mobility of the hole transport layer 120 and the electron mobility of the electron transport layer 140′ containing the above compound match. For example, the difference between the hole mobility of the hole transport layer 120 and the electron mobility of the electron transport layer 140′ of the above compound is within one order.

In the present embodiment, the light-emitting element 10 further includes a light-emitting layer 130. The light-emitting layer 130 is located between the hole transport layer 120 and the organic layer 140. The material of the light-emitting layer 130 may include an organic light-emitting material or a quantum dot light-emitting material, but the invention is not limited thereto. The quantum dot light-emitting material is, for example, cadmium selenide (CdSe). In the present embodiment, the light-emitting element 10 may further include an electron injection layer (not shown). The electron injection layer may be located between the organic layer 140 and the second electrode 150, but the invention is not limited thereto. In the present embodiment, the hole injection layer 110, the hole transport layer 120, the light-emitting layer 130, the organic layer 140, and the cathode 150 in the light-emitting element 10 may be formed by a method such as spin-coating, vacuum evaporation, or ink-jet printing (IP).

Based on the above, in the invention, the organic layer material in the light-emitting element includes the compound having the structure represented by formula (1) or the compound prepared by the method of synthesizing the compound having the pyridine ring, wherein the structure of the compound contains two fluorene rings connected to each other via a double bond such that the structure of the compound is flatter to achieve better molecular stacking. One carbon atom on each of the two benzene rings in one of the two fluorene rings is substituted by a nitrogen atom, such that the HOMO energy level and the LUMO energy level of the compound are reduced to reduce the energy barrier of electron injection. Therefore, the organic layer may have better electron mobility that is closer to the hole mobility in the hole transport layer, thus improving or ensuring the performance and life of the light-emitting element.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A compound having a structure represented by formula (1), 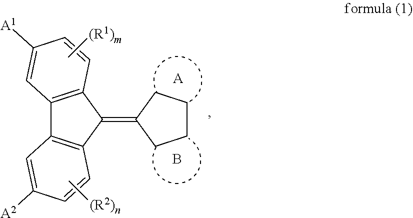
wherein ring A and ring B represent the same or different substituted or unsubstituted pyridine rings, respectively; A¹ and A² represent the same or different organic groups, respectively; R¹ and R² represent the same or different substituents, respectively; m and n represent 0, 1, 2 or 3, respectively.

2. The compound of claim 1, wherein formula (1) comprises a structure represented by formula (2): 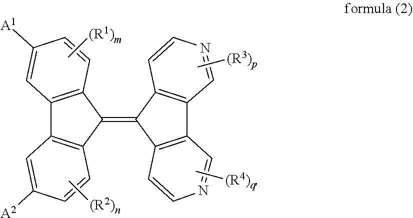
wherein A¹ and A² are the same or different organic groups; R¹, R², R³, and R⁴ are the same or different substituents, respectively; m, n, p and q are 0, 1, 2 or 3, respectively.

3. The compound of claim 1, wherein at least one of A¹ and A² has a structure represented by formula (3):
X—Y—* formula (3),
wherein X is a substituted or unsubstituted pyridine group or a substituted or unsubstituted pyrimidine group, Y is a substituted or unsubstituted divalent aromatic group, and * represents a bonding position.

4. The compound of claim 3, wherein X is 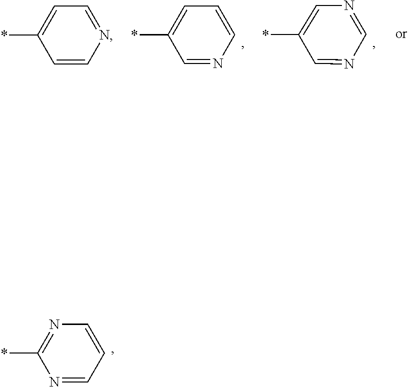
and * represents a bonding position.

5. The compound of claim 3, wherein Y is a substituted or unsubstituted phenylene group, a substituted or unsubstituted pyridylene group, or a substituted or unsubstituted pyrimidinylene group.

6. The compound of claim 1, wherein the compound having the structure represented by formula (1) is selected from one of the following compounds:

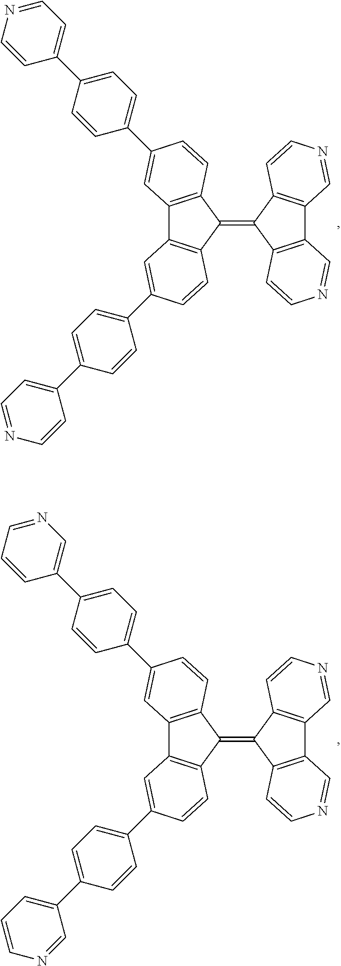 [SMILES: c1cc(-c2ccc(-c3ccc4c(c3)-c3cc(-c5ccc(-c6ccncc6)cc5)ccc3C4=C3c4ccncc4-c4cnccc43)cc2)ccn1 ; c1cncc(-c2ccc(-c3ccc4c(c3)-c3cc(-c5ccc(-c6cccnc6)cc5)ccc3C4=C3c4ccncc4-c4cnccc43)cc2)c1]

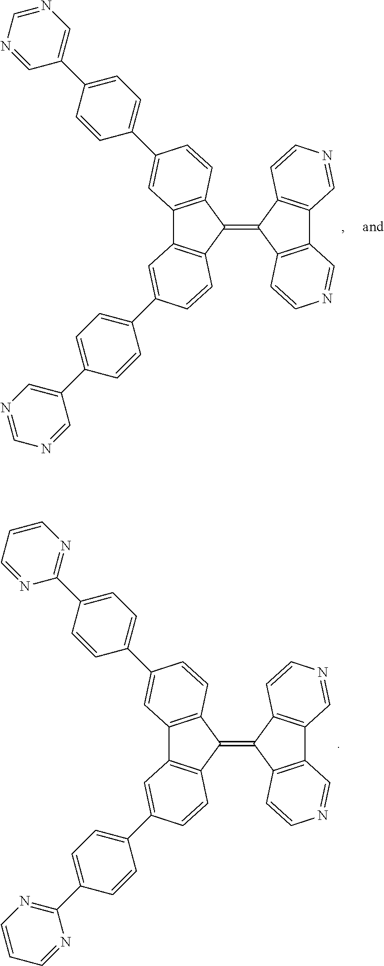 [SMILES: c1cc2c(cn1)-c1cnccc1C2=C1c2ccc(-c3ccc(-c4cncnc4)cc3)cc2-c2cc(-c3ccc(-c4cncnc4)cc3)ccc21 ; c1cnc(-c2ccc(-c3ccc4c(c3)-c3cc(-c5ccc(-c6ncccn6)cc5)ccc3C4=C3c4ccncc4-c4cnccc43)cc2)nc1]

7. The compound of claim 1, wherein formula (1) is used as a material of an electron transport layer, an electron mobility of the electron transport layer is between 1×10⁻⁴ cm²V⁻¹s⁻¹ and 9×10⁻⁴ cm²V⁻¹s⁻¹.

8. A method of synthesizing a compound with a pyridine ring, wherein the method comprises the following steps:
converting a 9,10-phenanthrenequinone to a borate ester compound; and
reacting the borate ester compound and at least one compound containing a halogen functional group to form the compound with the pyridine ring, wherein the borate ester compound has a structure represented by formula (1-1): 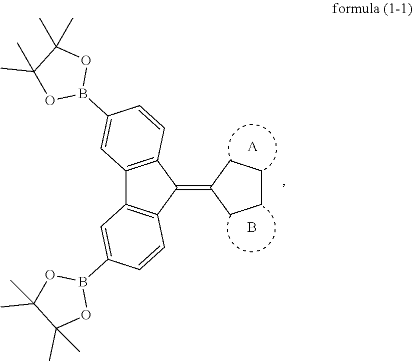
wherein ring A and ring B are the same or different substituted or unsubstituted pyridine rings, respectively.

9. The method of claim 8, wherein the step of converting the 9,10-phenanthrenequinone to the borate ester compound comprises:
converting the 9,10-phenanthrenequinone to a substituted or unsubstituted fluorenone; and
converting the substituted or unsubstituted fluorenone to the borate ester compound.

10. The method of claim 8, wherein the compound containing the halogen functional group comprises 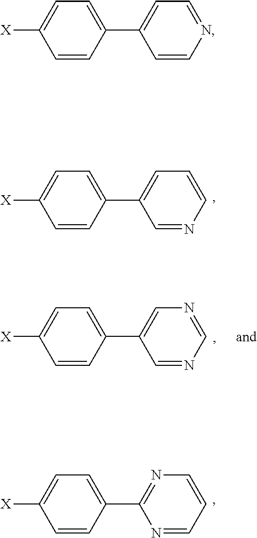
and N and X is a halogen atom.

11. A light-emitting element, comprising:
a first electrode;
an organic layer, wherein a material of the organic layer comprises the compound of claim 1; and
a second electrode,
wherein the organic layer is located between the first electrode and the second electrode.

12. The light-emitting element of claim 11, wherein the organic layer is used as a material of an electron transport layer.

13. The light-emitting element of claim 11, further comprising a hole transport layer, wherein the hole transport layer is located between the organic layer and the first electrode.

14. The light-emitting element of claim 13, wherein an electron mobility of the electron transport layer is between 1×10⁻⁴ cm²V⁻¹s⁻¹ and 9×10⁻⁴ cm²V⁻¹s⁻¹.

15. The light-emitting element of claim 13, wherein a material of the hole transport layer is poly(9,9′-dioctylfluorene-co-N-(4-butylphenyl) diphenylamine, poly(N-vinylcarbazole), or a combination thereof.

16. The light-emitting element of claim 11, further comprising a light-emitting layer, wherein the light-emitting layer is located between the organic layer and the first electrode.
